(19) United States
(12) Patent Application Publication
(10) Pub. No.: US 2016/0106545 A1
(43) Pub. Date: Apr. 21, 2016

(54) ARTIFICIAL IMPLANT FOR TRAPEZIOMETACARPAL JOINT

(71) Applicant: NATIONAL CHENG KUNG UNIVERSITY, TAINAN CITY (TW)

(72) Inventors: Wei-jr LIN, Tainan city (TW); I-ming JOU, Tainan city (TW); Che-chia HSU, Tainan city (TW); Fong-chin SU, Tainan city (TW)

(21) Appl. No.: 14/519,854

(22) Filed: Oct. 21, 2014

(51) Int. Cl. A61F 2/42

(52) U.S. Cl. CPC A61F 2/4261

(57) ABSTRACT

An artificial implant for a trapeziometacarpal joint is provided. The artificial implant has a joint replacement portion and at least two insertion rods. The insertion rods of the artificial implant can help to decrease the wound area during the implantation of the artificial implant in order to accelerate the speed of tendon reattachment after implanting and to enhance the ability of anti-rotation.

### FIELD OF THE INVENTION

The present invention relates to an artificial implant, and more particularly to an artificial implant for a trapeziometacarpal joint.

### BACKGROUND OF THE INVENTION

Osteoarthritis is a disease that affects the joints. When intra-articular cartilage of the joints is damaged or worn, it causes osteoarthritis, and thus causes joint stiffness, pain, and loss of function. Osteoarthritis commonly occurs in hip joints, knee joints, spinal joints, and joints of the hand (such as carpometacarpal joints). Among these, trapeziometacarpal (TMC) joint arthritis is one of the most important issues in the degenerative arthritis disease of the upper limbs. When medicaments or injections are not effective in relieving pain and can't further deal with the degenerative arthritis of the TMC joint, the injured TMC joint can be replaced by arthroplastic surgery, and thereby not only reducing pain from arthritis, but also maintaining TMC joint activities and the functions of the hand.

The process of a traditional surgery for replacing an artificial TMC joint is firstly to crosscut a wound from the root of the thumb, then release the attachment of the abductor pollicis longus (APL), open the joint capsule of the TMC joint, and abscise the damaged TMC joint surface at the proximal end of a first metacarpal to form an incision. Then, a portion of an implant is inserted into a bone marrow cavity of the first metacarpal from the incision at the proximal end of the first metacarpal. Thereafter, a surgical suture is used to reattach the APL tendon, followed by suturing the wound.

In Taiwan Application No. 201417788 entitled “ARTIFICIAL IMPLANT FOR CARPOMETACARPAL JOINT”, as shown in FIG. 1, an artificial implant 10 for a carpometacarpal (CMC) joint used for replacing a carpometacarpal joint articular surface of a metacarpal bone 11, the artificial implant 10 comprises an insert portion 12, and an articular replacement portion 13, wherein the insert portion 12 is used for inserting into a bone marrow cavity 14 exposed from the metacarpal bone 11. However, the insert portion 12 has one stem and uses press fit to fix on the bone. The stem length of the insert portion 12 is about the half that of the metacarpal bone 11, and a channel needs to be drilled from the bone marrow cavity 14 for matching the stem to fix the artificial implant 10. Furthermore, a width and a length of the surgical wound must be suitable for the artificial implant 10, so that the surgical wound is too large, and the time required to perform the surgery is increased.

As a result, it is necessary to provide an artificial implant for a trapeziometacarpal joint to solve the problems existing in the conventional technologies, as described above.

### SUMMARY OF THE INVENTION

A primary object of the present invention is to provide an artificial implant for trapeziometacarpal joint, which helps to decrease the wound area during implanting the artificial implant.

A secondary object of the present invention is to provide an artificial implant for a trapeziometacarpal joint, which provides an anti-torque moment by fixing the two insertion rods and the fin, and improves the friction of the insertion rods by forming a serrated surface.

Another object of the present invention is to provide an artificial implant for a trapeziometacarpal joint, the time required to perform the surgery is decreased by tying a suture on the abductor pollicis longus through the perforation.

To achieve the above objects, the present invention provides an artificial implant for a trapeziometacarpal joint, comprising a joint replacement portion formed with a connection surface for connecting to a cut end of a first bone and an articular profile surface for connecting to a joint surface of a second bone; and at least two insertion rods protruded from the connection surface and spaced from each other for inserting into two holes formed on the cut end of the first bone, wherein the connection surface has a long diameter and a short diameter, and the long diameter is perpendicular to the short diameter, and the length of each of the insertion rods is shorter than the short diameter.

In one embodiment of the present invention, the joint replacement portion further comprises at least one fin protruded from a lip portion of the connection surface for inserting into a groove formed on the cut end of the first bone.

In one embodiment of the present invention, the fin is equidistantly spaced from both of the two insertion rods.

In one embodiment of the present invention, the fin is provided on the lip portion of the connection surface at one end of the long diameter.

In one embodiment of the present invention, the joint replacement portion further comprises an outer peripheral surface delimited between the connection surface and the articular profile surface, and the outer peripheral surface has at least two suture perforations through which a suture passes.

In one embodiment of the present invention, each of the insertion rods has a serrated surface or a helical surface.

In one embodiment of the present invention, the diameter of each of the insertion rods is shorter than one half of the short diameter of the connection surface.

In one embodiment of the present invention, the insertion rods are provided on a right side and a left side of the long diameter of the connection surface, respectively.

In one embodiment of the present invention, the joint replacement portion and the insertion rods are made of biocompatible materials such as titanium, titanium alloy, polymer, cobalt-chromium-molybdenum alloy, or stainless steel.

In one embodiment of the present invention, the articular profile surface is saddle-shaped, double parabolic concave discoid, single parabolic concave discoid, spherical, hemispherical, polygonal spherical, or polygonal hemispherical.

In one embodiment of the present invention, the first bone is a first metacarpal bone, and the second bone is a trapezium.

As stated above, the present invention provides the insertion rods to insert into the cut end of the first bone, and the insertion rods of the artificial implant can help to decrease the size of the wound area during the implanting of the artificial implant in order to accelerate the speed of tendon reattachment after implanting and to enhance the ability of anti-rotation.

### DESCRIPTION OF THE DRAWINGS

FIG. 1 is an active condition view of a traditional artificial implant for a trapeziometacarpal joint;

FIG. 2 is a down perspective view of an artificial implant for a trapeziometacarpal joint according to a preferred embodiment of the present invention;

FIG. 3 is a top perspective view of an artificial implant for a trapeziometacarpal joint according to a preferred embodiment of the present invention; and

FIG. 4 is an active condition view of an artificial implant for a trapeziometacarpal joint according to a preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms used in the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

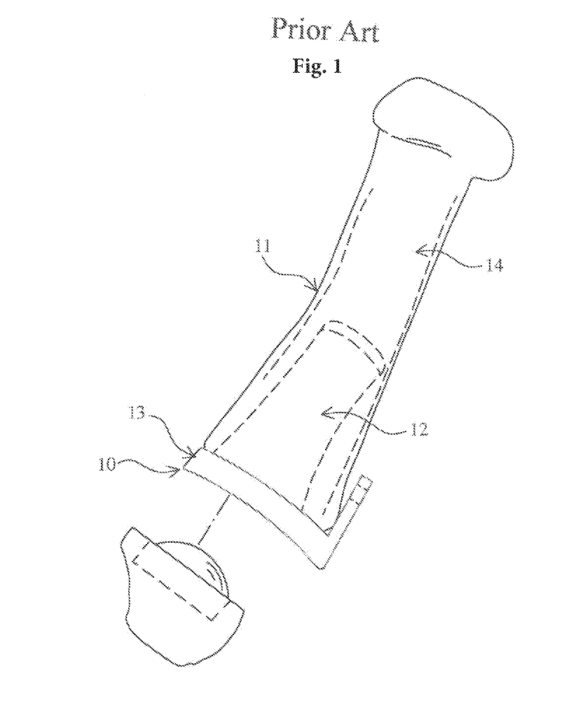

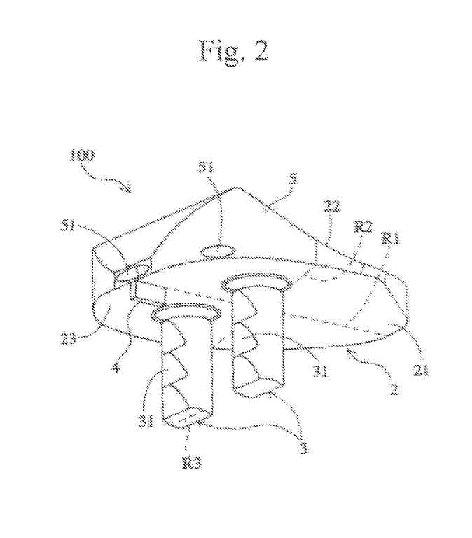

Referring now to FIGS. 1 and 2, wherein an artificial implant for a trapeziometacarpal joint according to a preferred embodiment of the present invention is illustrated. As shown, the artificial implant 100 for a trapeziometacarpal joint comprises a joint replacement portion 2, at least two insertion rods 3, at least one fin 4, and an articular profile surface 5. The insertion rods 3 are not restricted to two in the preferred embodiment, but also may be three or more in other embodiments. The joint replacement portion 2, the insertion rods 3, and the fin 4 are made of biocompatible materials, such as titanium, titanium alloy, polymer, cobalt-chromium-molybdenum alloy, or stainless steel, but are not limited thereto. The detailed structure of each component, assembly relationships and principle of operation in the present invention will be described in detail thereinafter.

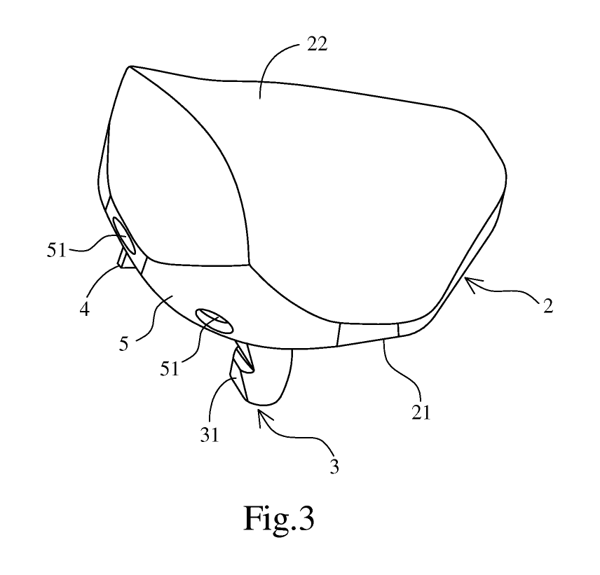

Referring now to FIGS. 2 to 3, in the preferred embodiment of the present invention, the joint replacement portion 2 forms with a connection surface 21 for connecting to a cut end of a first bone 101 (such as a first metacarpal bone) and an articular profile surface 22 for connecting to a joint surface of a second bone (such as a trapezium). The articular profile surface 22 is saddle-shaped, double parabolic concave discoid, single parabolic concave discoid, spherical, hemispherical, polygonal spherical, or polygonal hemispherical in the preferred embodiment, but is not limited thereto. In addition, the second bone can also be formed a cut, and the other artificial implant can be inserted into the cut.

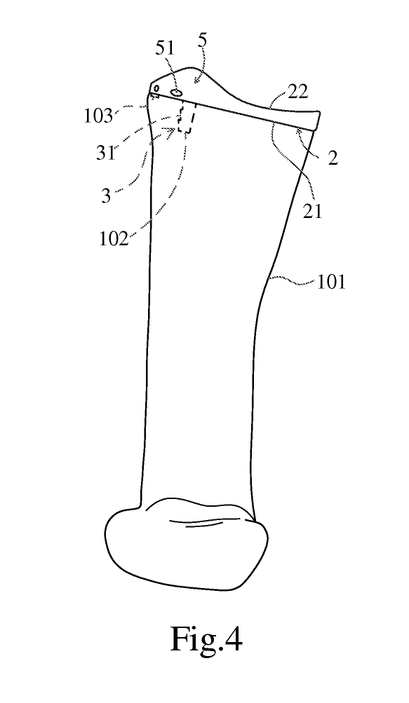

Referring still to FIGS. 2 to 4, in the preferred embodiment of the present invention, the insertion rods 3 are protruded from the connection surface 21 and spaced from each other, and each of the insertion rods 3 has a serrated surface 31 for inserting into two holes 102 formed on the cut end of the first bone 101. In addition, each of the insertion rods 3 also has a helical surface in other embodiments. The connection surface 21 has a long diameter R1 and a short diameter R2, and the long diameter R1 is perpendicular to the short diameter R2, and the insertion rods 3 are provided on a right side and a left side of the long diameter R1 of the connection surface 21, respectively. Furthermore, in the preferred embodiment, the length of each of the insertion rods 3 is shorter than that of the short diameter R2, and the diameter of each of the insertion rods 3 is shorter than one half of the short diameter R2 of the connection surface 21.

Referring now to FIGS. 2 to 4, in the preferred embodiment of the present invention, the outer peripheral surface 5 is delimited between the connection surface 21 and the articular profile surface 22, and the outer peripheral surface 5 has at least two suture perforations 51. In the preferred embodiment, there are two suture perforations 51 through which a suture passes for connecting a suture onto the abductor pollicis longus on the bone.

Referring now to FIGS. 2 to 4, when a surgery for replacing an artificial TMC joint is underway and using press fit to fix on the bone. First, channels are drilled from the bone marrow cavity of the first bone 101, and are matched with the insertion rods 3, and then the insertion rods 3 of the artificial implant 100 are inserted into the channels, so that the connection surface 21 is attached to the cut end of the first bone 101, and the fin 4 is inserted into the groove 103, and the artificial implant 100 can be connected to the first bone 101.

As stated above, the present invention provides the insertion rods 3 to insert into the cut end of the first bone 101, and the insertion rods 3 of the artificial implant 100 can help to decrease the area of the wound during the implantation of the artificial implant, to accelerate the speed of tendon reattachment after implanting, and to enhance the ability of anti-rotation. Furthermore, an anti-torque moment can be provided by fixing the two insertion rods and the fin, and the friction of the insertion rods can be improved by forming a serrated surface, and the operative time can be reduced by tying a suture onto the abductor pollicis longus through the perforation.

The present invention has been described with a preferred embodiment thereof and it is understood that many changes and modifications to the described embodiment can be carried out without departing from the scope and the spirit of the invention that is intended to be limited only by the appended claims.

1. An artificial implant for a trapeziometacarpal joint, comprising:
a joint replacement portion formed with a connection surface for connecting to a cut end of a first bone and an articular profile surface for connecting to a joint surface of a second bone; and
at least two insertion rods protruded from the connection surface and spaced from each other for inserting into two holes formed on the cut end of the first bone, wherein the connection surface has a long diameter and a short diameter, and the long diameter is perpendicular to the short diameter, and the length of each of the insertion rods is shorter than the short diameter; and
at least one fin protruded from an edge portion of the connection surface for inserting into a groove formed on the cut end of the first bone, wherein the fin is equidistantly spaced from both of the two insertion rods, and the fin is provided on the edge portion of the connection surface at one end of the long diameter.

2-4. (canceled)

5. The artificial implant according to claim 1, wherein the joint replacement portion further comprises an outer peripheral surface delimited between the connection surface and the articular profile surface, and the outer peripheral surface has at least two suture perforations through which a suture passes.

6. The artificial implant according to claim 1, wherein each of the insertion rods has a serrated surface or a helical surface.

7. The artificial implant according to claim 1, wherein the diameter of each of the insertion rods is shorter than one half of the short diameter of the connection surface.

8. The artificial implant according to claim 7, wherein the insertion rods are provided on a right side and a left side of the long diameter of the connection surface, respectively.

9. The artificial implant according to claim 1, wherein the joint replacement portion and the insertion rods are made of biocompatible materials selected from the group consisting of titanium, titanium alloy, polymer, cobalt-chromium-molybdenum alloy, and stainless steel.

10. The artificial implant according to claim 1, wherein the articular profile surface is saddle-shaped, double parabolic concave discoid, single parabolic concave discoid, spherical, hemispherical, polygonal spherical, or polygonal hemispherical.

11. The artificial implant according to claim 1, wherein the first bone is a first metacarpal bone, and the second bone is a trapezium.
